(19) United States
(12) Patent Application Publication
(10) Pub. No.: US 2008/0140005 A1
(43) Pub. Date: Jun. 12, 2008

(54) Disposable safety syringe

(75) Inventors: Songneng Luo, Puning (CN); Shaoji Luo, Puning (CN)

Correspondence Address: Raymond Y. Chan, Suite 128, 108 N. Ynez Avenue, Monterey Park, CA, 91754, US

(73) Assignee: Songneng LUO

(21) Appl. No.: 11/998,010

(22) Filed: Nov. 27, 2007

(30) Foreign Application Priority Data
Dec. 6, 2006 (CN) 200620154651.7
May 3, 2007 (CN) 200720051269.8
Oct. 25, 2007 (CN) 200710031084.5

(51) Int. Cl. A61M 5/32

(52) U.S. Cl. 604/110; 604/218

(57) ABSTRACT

A disposable safety syringe includes a barrel body, a needle seat holding a needle in position, a plunger body, having an interior cavity, slidably mounted in the barrel body, and a needle retraction device including an end stopper, having a retraction ring, mounted in the plunger body, a compression spring mounted at the end stopper, and a retraction head integrally downwardly and integrally protruding from a bottom end of the needle seat to align with the retraction ring, wherein when the plunger body is kept moving towards the upper portion of the barrel body after the injection operation is completed, the retraction head is engaged with the retraction ring to couple the needle seat with the end stopper, such that the compression spring pushes the end stopper into the plunger body to retract the needle seat and the needle into the plunger body.

### BACKGROUND OF THE PRESENT INVENTION

1. Field of Invention

The present invention relates to a syringe, and more particularly to a disposable safety syringe for one time use that the needle is retracted into the barrel after the use of the syringe.

2. Description of Related Arts

Disposable syringes are widely used in medical area for avoiding cross-infection of diseases in those years. In order to prevent the disposable syringes being reused, self destroying structures are developed for the syringe. However, even though the self destroying structure can reduce the chance of cross-infection of diseases owing to its self destroy structure, it still has many drawbacks. The exposed steel needles of the used syringe still cause a lot of trouble during the collecting, transporting, and disposing. Moreover, the people dealing with the used syringes still have the risk of being injured.

The Chinese patent has disclosed a needle retractable safety syringe. In that invention, the syringe comprises a compression spring between the needle seat and the upper portion of the injector. When the medication is injected through the needle, the spring retracts the needle seat with the needle together into the barrel. This invention realized self destroy to prevent the needle being exposed out of the barrel to hurt people for safety purpose. However, the structure of the injector is relatively complicated and hard to assemble during manufacturing.

### SUMMARY OF THE PRESENT INVENTION

The major object of the present invention is to provide a disposable safety syringe which is easy to manufacture. Also it is suitable for mass product by machine manufacturing and assembly line.

In order to accomplish the above object, the present invention provides a disposable safety syringe comprising a needle made of steel, a needle seal, a barrel body, and a plunger body slidably mounted inside the barrel body. The needle is held by the needle seat. The needle seat comprises a protruding ring outwardly and radially protruding from the bottom portion of the needle seat. The ring-shaped gasket is mounted inside the barrel body at a position between an outer wall of the protruding ring and an inner wall of the barrel body. The plunger body has an elongated hollow structure. A compression spring is mounted in the plunger body. A piston is mounted at the upper portion of the plunger body. The upper portion of the plunger body forms a size-reducing portion. An end stopper is mounted in the interior cavity of the plunger body. The plunger body further has an engaging groove provided at an inner wall of the size-reducing portion of the plunger body, wherein the end stopper has an engaging ring outwardly and radially protruding from an outer wall of the end stopper to engage with the engaging groove to retain the end stopper at the size-reducing portion of the plunger body. A needle retraction device is provided between the end stopper and the bottom portion of the needle seat for retracting the needle seat after the medication injection.

According to the second preferred embodiment of the present invention, the disposable safety syringe comprises a needle made of steel, a needle seat, a barrel body, and a plunger body slidably mounted inside the barrel body. The needle seat is retained at an outer needle sleeve to rotatably mount to the barrel body via a threaded structure. An engaging groove is radially indented on an inner wall of the needle sleeve while an engaging ring is radially protruded from an inner wall of the needle seat to engage with the engaging groove so as to retain the needle seat within the needle sleeve. Accordingly, the plunger body has an elongated hollow structure. A compression spring is mounted in the plunger body. An elastic holder is mounted at the upper portion of the plunger body. The upper portion of the plunger body forms a size-reducing portion. An end stopper is mounted in the interior cavity of the plunger body. The plunger body further has an engaging groove provided at an inner wall of the size-reducing portion of the plunger body, wherein the end stopper has an engaging ring outwardly and radially protruding from an outer wall of the end stopper to engage with the engaging groove to retain the end stopper at the size-reducing portion of the plunger body. A needle retraction device is provided between the end stopper and the bottom portion of the needle seat for retracting the needle seat after the medication injection.

The needle retraction device comprises a hooking shaped retraction head downwardly and integrally protruding from a bottom end of the needle seat and a retraction ring provided at a top end of the end stopper to align with the retraction head, and a compression spring coaxially mounted at the lower portion of the end stopper for applying an urging force against the plunger body.

The piston can be an elastic holder which comprises a lower holding ring, a middle holding ring, and an upper holding ring. The lower holding ring is integrally extended from the middle holding ring, wherein the upper holding ring is extended from the middle holding ring via an elastic compression ring. An indentation groove is formed at an inner wall of the middle holding ring to engage with the size-reducing portion of the plunger body. The elastic holder applied in this needle retractable self destroy safety syringe can reach the “0” calibration without changing the structure of the syringe. The remaining medication in the barrel satisfies the quality requirement.

Finally, the structure of the present invention is novelty, and simple. The requirement of the assembly process is low. Furthermore, it is suitable for mass product by machine manufacturing and assembly line.

These and other objectives, features, and advantages of the present invention will become apparent from the following detailed description, the accompanying drawings, and the appended claims.

### BRIEF DESCRIPTION OF THE DRAWINGS

FIG. 1 is a sectional view of a disposable safety syringe of a first preferred embodiment of the present invention.

FIG. 2 is a sectional view of the disposable safety syringe according to the above first preferred embodiment of the present invention, illustrating the needle being retracted in the barrel body.

FIG. 3 is a sectional view of an elastic holder of a disposable safety syringe according to a second preferred embodiment of the present invention.

FIG. 4 is a sectional view of the disposable safety syringe according to the second preferred embodiment of the present invention.

FIG. 5 is a sectional view of the disposable safety syringe according to a third preferred embodiment of the present invention.

FIG. 6 is a sectional view of the disposable safety syringe according to the third preferred embodiment of the present invention, illustrating the elastic holder being compressed.

FIG. 7 is a sectional view of the disposable safety syringe according to the third preferred embodiment of the present invention, illustrating the needle being retracted in the barrel body.

### DETAILED DESCRIPTION OF THE PREFERRED EMBODIMENT

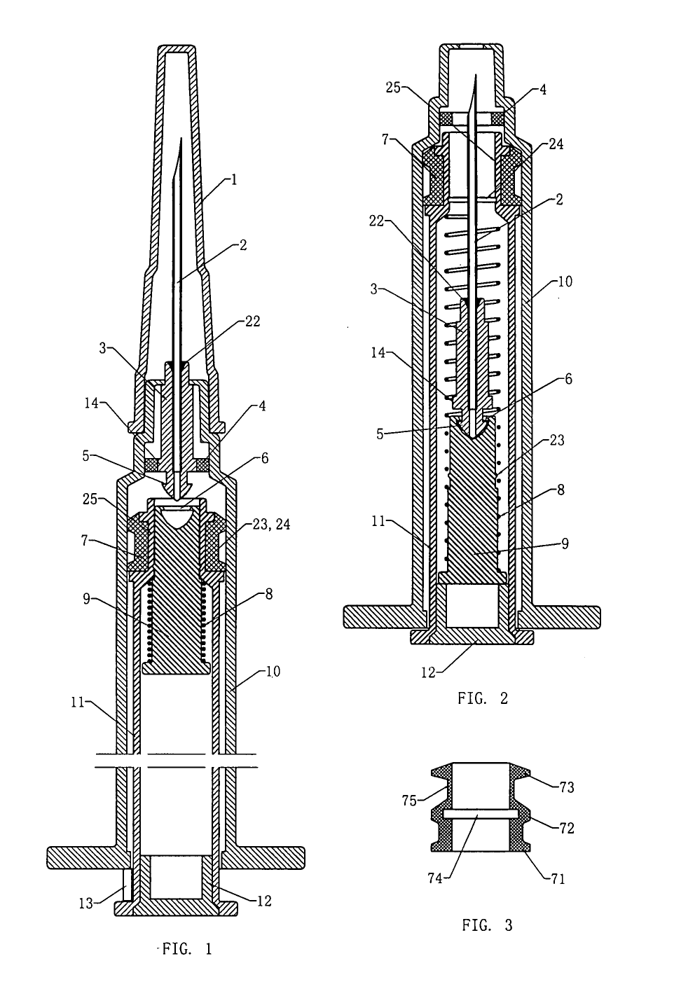

Referring to FIGS. 1 to 2 of the drawings, a disposable safety syringe according to a preferred embodiment of the present invention is illustrated, in which the disposable safety syringe comprises a needle cap 1, a needle 2 made of steel, a needle seat 3, a barrel body 10, and a plunger body 11 slidably mounted in the barrel body 10. The needle 2 is held at an upper portion of the needle seat 3. The needle seal 3 also comprises a sealing chamber 22. When the needle 2 is extended out from the needle seat 3, glue or other sealing element can be filled inside the sealing chamber 22 for sealing and retaining the needle 2 in position. The needle seat 3 comprises a protruding ring 14 outwardly and radially protruding from the bottom portion of the needle seat 3. The ring-shaped gasket 4 is mounted inside the barrel body 10 at a position between an outer wall of the protruding ring 14 and an inner wall of the barrel body 10, wherein the gasket 4 is made of elastic material for applying an elastic force between the barrel body 10 and the protruding ring 14 to retain the needle seat 3 in position. The gasket 4 also let the needle seat 3 being retracted into the barrel body 10 once the elastic force is released. In other words, the gasket 4 form a part of a needle retraction structure of the present invention.

The plunger body 11 has an elongated hollow structure to define an interior cavity, wherein a compression spring 11 is disposed in the interior cavity of the plunger body 11. An elastic piston 7 is mounted at the upper portion of the plunger body 11 while a plug 12 is mounted at the bottom portion of the plunger body 11. Accordingly, the upper portion of the plunger body 11 forms a size-reducing portion 25 for the elastic piston 7 mounted therearound, wherein the size-reducing portion 25 has a diameter smaller than a diameter of the bottom portion of the plunger body 11. An end stopper 9 is mounted in the interior cavity of the plunger body 11 at a position that an upper portion of the end stopper 9 is mounted within the size-reducing portion 25 while a lower portion of the end stopper 9 is extended towards the bottom portion of the plunger body 11. The plunger body 11 further has an engaging groove 24 provided at an inner wall of the size-reducing portion 25 of the plunger body 11, wherein the end stopper 9 has an engaging ring 23 outwardly and radially protruding from an outer wall of the upper portion of the end stopper 9 to engage with the engaging groove 24 to normally retain the end stopper 9 in position. Accordingly, when the engaging ring 23 is engaged with the engaging groove 24, the engagement therebetween will prevent the needle seat 3 from being retracted accidentally especially during the medication injection. Thus, it provides the sealing effect of the plunger body 11. A needle retraction device is provided between the end stopper 9 and the bottom portion of the needle seal 3 for retracting the needle seal 3 after the medication injection. The needle retraction device comprises a hooking shaped retraction head 5 downwardly and integrally protruding from a bottom end of the needle seat 3 and a retraction ring 6 provided at a top end of the end stopper 9 to align with the retraction head 5, and a compression spring 8 coaxially mounted at the lower portion of the end stopper 9 for applying an urging force against the plunger body 11.

In order to use the syringe of the present invention, a locking ring 13 must be firstly removed from the plunger body 11. Therefore, the user is able to the syringe is adapted for suck the liquid into the barrel body 10 when the plunger body 11 is downwardly slid within the barrel body 10. Then, the plunger body 11 is slid to press the liquid out of the barrel body 10 through the needle 2. The liquid suction and injection operation of the syringe of the present invention is the same as that of the conventional syringe. After the injection of the syringe, the plunger body 11 is kept being pressed at the injection direction until the retraction head 5 is engaged with the retraction ring 6 so as to couple the plunger body 11 with the needle seat 3. When the plunger body 11 is kept being pressed forward, i.e. the injection direction, the gasket 4 is pushed to slide forward within the barrel body 10. Once forward movement of the gasket 4 is blocked and the elastic piston 7 is compressed for stopping the further forward movement of the plunger body 11, the end stopper 9 is driven downwardly. Therefore, the engaging ring 23 will be disengaged with the engaging groove 24 within the size-reducing portion 25 of the plunger body 11. Once the frictional engagement between engaging ring 23 and the engaging groove 24 is released, the compressed compression spring 8 is bounded to its original form that the end stopper 9, the needle seat 3, and the needle 2 are retracted into the interior cavity of the plunger body 11 via the spring force. Therefore, the syringe of the present invention cannot be reused after the injection by the destroying structure of the syringe. Finally, the used needle 2 and liquid of medication will be kept inside the plunger body 1 I to enhance the safety purpose of the syringe.

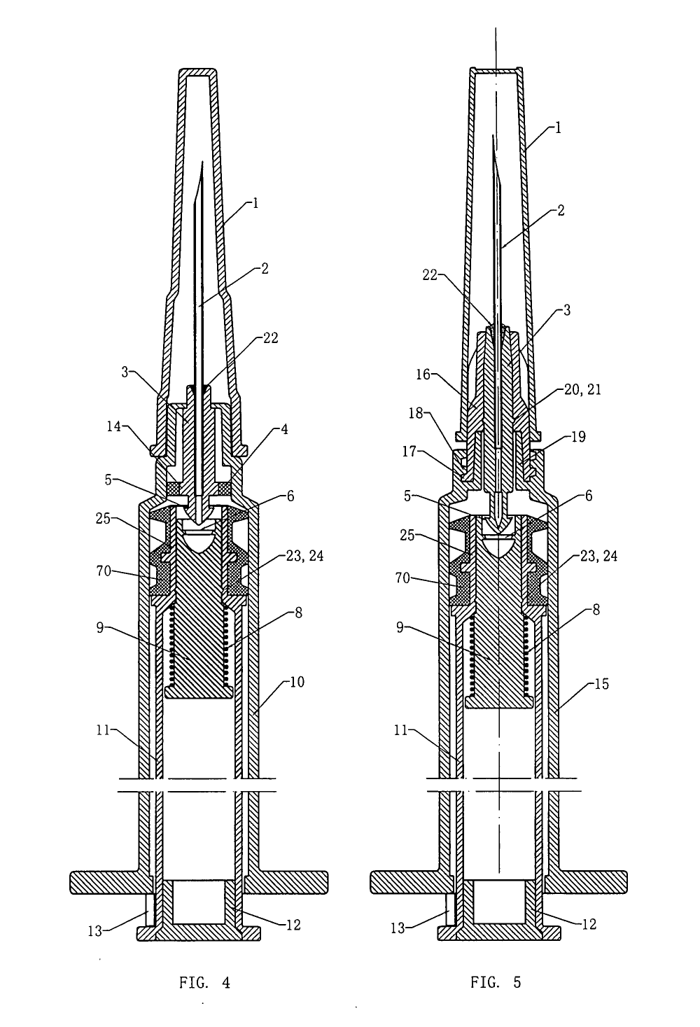

Referring to FIGS. 3 and 4 of the drawings, a disposable safety syringe of a second embodiment illustrates an alternative mode of the first embodiment, wherein the difference between the first and second embodiments is that the piston 7 of the first embodiment forms a tubular elastic holder 70 in the second embodiment. According to the second embodiment, the elastic holder 70 comprises a lower holding ring 71, a middle holding ring 72, and an upper holding ring 73. The lower holding ring 71 is integrally extended from the middle holding ring 72, wherein the upper holding ring 73 is extended from the middle holding ring 72 via an elastic compression ring 75. An indentation groove 74 is formed at an inner wall of the middle holding ring 72 to engage with the size-reducing portion 25 of the plunger body 11. The operation of the syringe of the second embodiment is the same as that of the first embodiment.

Referring to FIGS. 5 to 7 of the drawings, a disposable safety syringe according to a third embodiment of the present invention is illustrated as an alternative mode of the second embodiment. Accordingly, the syringe further comprises an outer needle sleeve 16 holding the needle seat 3 therewithin, wherein the needle sleeve 16 is mounted to the barrel body 15. Accordingly, the needle sleeve 16 has an outer threaded portion 17. The barrel body 15 further comprises a sleeve holder 19, having an inner threaded portion 18, integrally formed at the upper portion of the barrel body 15, wherein the outer threaded portion 17 of the needle sleeve 16 is rotatably engaged with the inner threaded portion 18 of the sleeve holder 19 to mount the needle seat 3 at the barrel body 15.

The needle retraction device is mounted at the needle seat 3, wherein the needle retraction device further contains a retention groove 21 radially indent on an inner wall of the needle sleeve 16 and comprises a retention ring 20 radially protruded from an outer wall of the needle seat 3 to engage with the retention groove 21 so as to retain the needle seat 3 within the needle sleeve 16. In other words, the engagement between the retention groove 21 and the retention ring 20 prevents the needle 2 from being retracted academically during injection and ensures the sealing effect of the interior of the barrel body 15 to contain the liquid therein. Thus, it is suitable for mass product by machine manufacturing and assembly line.

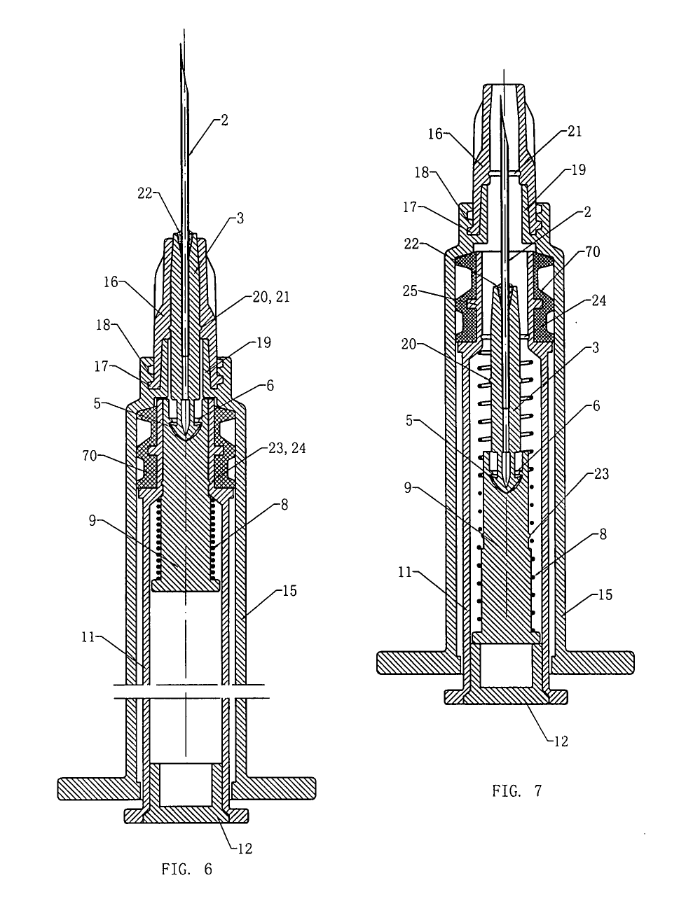

In order to use the syringe, the locking ring 1 3 must be firstly removed from the plunger body 11. Therefore, the user is able to the syringe is adapted for suck the liquid into the barrel body 15 when the plunger body 11 is downwardly slid within the barrel body 15. Then, the plunger body 11 is slid to press the liquid out of the barrel body 15 through the needle 2. The liquid suction and injection operation of the syringe of the present invention is the same as that of the conventional syringe. After the injection of the syringe, the plunger body 11 is kept being pressed at the injection direction until the retraction head 5 is engaged with the retraction ring 6 so as to couple the plunger body 11 with the needle seal 3. When the plunger body 11 is kept being pressed forward, i.e. the injection direction, the elastic holder 70 is compressed to store the compression force, as shown in FIG. 6. On the other hand, the end stopper 9 is pushed downwardly, i.e. the opposite direction of the injection direction, by the reaction force of the needle seat 3. Once the total compression forces of the elastic holder 70 and the compressed compression spring 8 is larger than the engagement force between the retention groove 21 and the retention ring 20, the total compression forces will push the needle seat 3 and the needle 2 into the interior cavity of the plunger body 11, as shown in FIG. 7. Therefore, the syringe of the present invention cannot be reused after the injection by the destroying structure of the syringe. Finally, the used needle 2 and liquid of medication will be kept inside the plunger body 11 to enhance the safely purpose of the syringe.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A disposable safety syringe, comprising:
a barrel body for containing a predetermined liquid therein;
a needle seat holding a needle in position, wherein said needle seat is mounted at an upper portion of said barrel body;
a plunger body, having an interior cavity, slidably mounted in said barrel body, wherein said plunger body is slidably moved towards said upper portion of said barrel body for injecting said liquid through said needle so as to complete an injection operation; and
a needle retraction device, which comprises:
an end stopper mounted in said interior cavity of said plunger body, wherein said end stopper comprises a retraction ring formed at a top side of said end stopper;
a compression spring mounted at said end stopper for applying an urging force against said plunger body; and
a retraction head integrally downwardly and integrally protruding from a bottom end of said needle seat to align with said retraction ring, wherein when said plunger body is kept moving towards said upper portion of said barrel body after said injection operation is completed, said retraction head is engaged with said retraction ring to couple said needle seat with said end stopper, such that said compression spring pushes said end stopper into said interior cavity of said plunger body to retract said needle seat and said needle into said plunger body.

2. The disposable safety syringe, as recited in claim 1, wherein said plunger body has an engaging groove provided at an inner wall of said plunger body, wherein said end stopper has an engaging ring outwardly and radially protruding from an outer wall of said end stopper to engage with said engaging groove to normally retain said end stopper in position, such that when said plunger body is blocked for further forward movement, said engaging ring is disengaged with said engaging groove to allow said end stopper being retracted in said plunger body via said compression spring.

3. The disposable safety syringe, as recited in claim 1, wherein said upper portion of said plunger body forms a size-reducing portion that said size-reducing portion has a diameter smaller than a diameter of a bottom portion of said plunger body, wherein an upper portion of said end stopper is mounted within said size-reducing portion while a lower portion of said end stopper is extended towards said bottom portion of said plunger body.

4. The disposable safety syringe, as recited in claim 2, wherein said upper portion of said plunger body forms a size-reducing portion that said size-reducing portion has a diameter smaller than a diameter of a bottom portion of said plunger body, wherein an upper portion of said end stopper is mounted within said size-reducing portion while a lower portion of said end stopper is extended towards said bottom portion of said plunger body.

5. The disposable safety syringe, as recited in claim 3, wherein said compression spring is mounted at said lower portion of said end stopper.

6. The disposable safety syringe, as recited in claim 4, wherein said compression spring is mounted at said lower portion of said end stopper.

7. The disposable safety syringe, as recited in claim 5, wherein said engaging groove is provided at said inner wall of said plunger body at said size-reducing portion thereof, wherein said engaging ring is outwardly and radially protruding from said outer wall of said end stopper at said upper portion thereof to engage with said engaging groove.

8. The disposable safety syringe, as recited in claim 6, wherein said engaging groove is provided at said inner wall of said plunger body at said size-reducing portion thereof, wherein said engaging ring is outwardly and radially protruding from said outer wall of said end stopper at said upper portion thereof to engage with said engaging groove.

9. The disposable safety syringe, as recited in claim 7, further comprising an elastic piston mounted around said size-reducing portion of said plunger body to block a further forward movement of said plunger body.

10. The disposable safety syringe, as recited in claim 8, further comprising an elastic piston mounted around said size-reducing portion of said plunger body to block a further forward movement of said plunger body.

11. The disposable safety syringe, as recited in claim 10, further comprising a gasket sealed and mounted at said upper portion of said barrel body at a position between an outer wall of said needle seat and an inner wall of the barrel body.

12. The disposable safety syringe, as recited in claim 7, further comprising a tubular elastic holder mounted around said size-reducing portion of said plunger body to block a further forward movement of said plunger body, wherein said elastic holder comprises a lower holding ring, a middle holding ring integrally extended from said lower holding ring, and an upper holding ring extended from said middle holding ring via an elastic compression ring, wherein said elastic holder has an indentation groove formed at an inner wall of said middle holding ring to engage with said size-reducing portion of said plunger body.

13. The disposable safety syringe, as recited in claim 8, further comprising a tubular elastic holder mounted around said size-reducing portion of said plunger body to block a further forward movement of said plunger body, wherein said elastic holder comprises a lower holding ring, a middle holding ring integrally extended from said lower holding ring, and an upper holding ring extended from said middle holding ring via an elastic compression ring, wherein said elastic holder has an indentation groove formed at an inner wall of said middle holding ring to engage with said size-reducing portion of said plunger body.

14. The disposable safety syringe, as recited in claim 12, further comprising an outer needle sleeve holding said needle seat therewithin, wherein said needle sleeve is rotatably mounted to said barrel body to retain said needle seat in position.

15. The disposable safety syringe, as recited in claim 13, further comprising an outer needle sleeve holding said needle seal therewithin, wherein said needle sleeve is rotatably mounted to said barrel body to retain said needle seal in position.

16. The disposable safety syringe, as recited in claim 13, wherein said needle retraction device further contains a retention groove radially indent on an inner wall of said needle sleeve and comprises a retention ring radially protruded from an outer wall of said needle seat to engage with said retention groove so as to retain said needle seat within said needle sleeve.

17. The disposable safety syringe, as recited in claim 13, wherein said needle retraction device further contains a retention groove radially indent on an inner wall of said needle sleeve and comprises a retention ring radially protruded from an outer wall of said needle seat to engage with said retention groove so as to retain said needle seat within said needle sleeve.

18. The disposable safety syringe, as recited in claim 1, wherein said retraction head has a hook shape to engage with said retraction ring so as to couple said needle seat with said end stopper.

19. The disposable safety syringe, as recited in claim 11, wherein said retraction head has a hook shape to engage with said retraction ring so as to couple said needle seat with said end stopper.

20. The disposable safety syringe, as recited in claim 17, wherein said retraction head has a hook shape to engage with said retraction ring so as to couple said needle seat with said end stopper.
